(19) United States
(12) Patent Application Publication
(10) Pub. No.: US 2026/0183006 A1
(43) Pub. Date: Jul. 2, 2026

(54) THROMBUS REMOVAL SYSTEMS AND ASSOCIATED METHODS

(71) Applicant: Shifamed Holdings, LLC, Campbell, CA (US)

(72) Inventor: Tom SAUL, Portland, OR (US)

(21) Appl. No.: 18/867,656

(22) PCT Filed: May 22, 2023
(86) PCT No.: PCT/US2023/067299

§ 371 Date: Nov. 20, 2024

Related U.S. Application Data
(60) Provisional application No. 63/365,093, filed on May 20, 2022.

(51) Int. Cl. A61B 17/221; A61B 17/22

(52) U.S. Cl. CPC A61B 17/221; A61B 2017/22051; A61B 2017/22062; A61B 2017/22065; A61B 2017/22079; A61B 2017/2215

(57) ABSTRACT

The present technology relates to systems and methods for removing a thrombus from a blood vessel of a patient. In some embodiments, the present technology is directed to systems including an elongated catheter having a distal portion configured to be positioned within the blood vessel of the patient, a proximal portion configured to be external to the patient, and a lumen extending therebetween. The system can also include a fluid delivery mechanism coupled with a fluid lumen and configured to apply fluid to at least partially fragment the thrombus, and an aspiration mechanism fluidly coupled to an aspiration lumen and configured to aspirate the fragmented thrombus.

### CLAIM OF PRIORITY

This application claims the benefit of U.S. Provisional Application No. 63/365,093, filed May 20, 2022, which is herein incorporated by reference in its entirety for all purposes.

### RELATED APPLICATIONS

This application is related to International Application No. PCT/US2021/020915, filed Mar. 4, 2021, U.S. Application No. 63/190,784, filed May 19, 2021, and U.S. Application No. 63/335,656, filed Apr. 27, 2022, the disclosures of which are incorporated by reference herein.

### INCORPORATION BY REFERENCE

All publications and patent applications mentioned in this specification are herein incorporated by reference to the same extent as if each individual publication or patent application was specifically and individually indicated to be incorporated by reference.

### FIELD

The present technology generally relates to medical devices and, in particular, to systems including aspiration and fluid delivery mechanisms and associated methods for removing a thrombus from a mammalian blood vessel.

### BACKGROUND

Thrombotic material may lead to a blockage in fluid flow within the vasculature of a mammal. Such blockages may occur in varied regions within the body, such as within the pulmonary system, peripheral vasculature, deep vasculature, or brain. Pulmonary embolisms typically arise when a thrombus originating from another part of the body (e.g., a vein in the pelvis or leg) becomes dislodged and travels to the lungs. Anticoagulation therapy is the current standard of care for treating pulmonary embolisms, but may not be effective in some patients. Additionally, conventional devices for removing thrombotic material may not be capable of navigating the vascular anatomy of the lungs, may not be effective in removing thrombotic material, and/or may lack the ability to provide sensor data or other feedback to the clinician during the thrombectomy procedure.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIGS. 1-1A illustrate various views of a portion of a thrombus removal system including a distal portion of an elongated catheter configured in accordance with an embodiment of the present technology.

FIGS. 2A-2F illustrate various views of one or more inflatable member(s) of a funnel of a thrombus removal device.

FIGS. 3A-3D illustrate additional top-down views of one or more inflatable member(s) of a funnel of a thrombus removal device.

FIGS. 4A-4B show one embodiment of a thrombus removal device having a funnel and one or more inflatable member(s) and scraping elements.

FIGS. 5A-5C show an example of a thrombus removal device having a funnel and one or more inflatable member(s).

FIGS. 6A-6D show embodiments of a thrombus removal device having a funnel and one or more inflatable members or lumens configured to provide jets or fluid streams into the funnel.

### SUMMARY OF THE DISCLOSURE

A thrombus removal system, comprising an elongated catheter device including a distal portion configured to be positioned within a blood vessel of a patient, the distal portion including a fluid lumen, a funnel positioned distal to the distal portion and configured to engage a thrombus and/or a vessel wall, at least two fluid ports formed in the distal portion or in the funnel and adapted for fluid communication with the fluid lumen to direct respective fluid streams from the at least two fluid ports, at least one inflatable member positioned on or in the funnel, the at least one inflatable member being configured to improve engagement with a thrombus and/or a vessel wall, a proximal portion configured to be positioned external to the patient, the aspiration lumen extending from the distal portion to the proximal portion, an aspiration mechanism positioned external to the patient and fluidly coupled with the aspiration lumen, the aspiration mechanism configured to reduce a pressure at the distal portion (a) to engage a thrombus therewith and/or (b) to draw the thrombus and/or thrombus fragments proximally, and a fluid delivery mechanism configured to supply fluid through the fluid lumen.

In some embodiments, the at least one inflatable member is disposed on an interior surface of the funnel.

In one example, the at least one inflatable member is disposed on an exterior surface of the funnel.

In some embodiments, the at least one inflatable member extends along a periphery of the funnel.

In some examples, the at least one inflatable member comprises discrete inflatable members disposed at selected positions along the funnel.

In one embodiment, the at least one inflatable member has a partially circular cross-sectional shape.

In some embodiments, the at least one inflatable member has a generally rectangular cross-sectional shape.

In one embodiment, the at least one inflatable member is configured to be inflated with a fluid, a gas, or a vapor.

A method for removing a thrombus from a blood vessel of a patient is provided, the method comprising introducing a distal portion of an elongate catheter in proximity to a thrombus location in a blood vessel, expanding a funnel distal to the distal portion in the blood vessel, drawing at least a section of the thrombus into the distal portion, expanding at least one inflatable member of the funnel to improve engagement between the thrombus and the funnel, and directing fluid toward the thrombus with at least two different fluid streams.

A method for removing a thrombus from a blood vessel of a patient, the method comprising introducing a distal portion of an elongate catheter in proximity to a thrombus location in a blood vessel, expanding a funnel distal to the distal portion in the blood vessel, drawing at least a section of the thrombus into the distal portion, expanding at least one inflatable member of the funnel to improve engagement between a vessel wall and the funnel, and directing fluid toward the thrombus with at least two different fluid streams.

In one aspect, a thrombus removal device is provided, comprising an elongate catheter shaft, an aspiration lumen disposed in the elongate catheter shaft, a funnel positioned at a distal portion of the elongate catheter shaft and configured to engage a thrombus and/or a vessel wall, at least two fluid ports formed in the distal portion and adapted to direct fluid streams from the at least two fluid ports, at least one inflatable member positioned on or in the funnel, the at least one inflatable member being configured to improve engagement with the thrombus and/or vessel wall.

In some aspects, the at least one inflatable member is disposed on an interior surface of the funnel. In one aspect, the at least one inflatable member is disposed on an exterior surface of the funnel. In another aspect, the at least one inflatable member extends along a periphery of the funnel.

In some aspects, the at least one inflatable member comprises discrete inflatable members disposed at selected positions along the funnel.

In one aspect, the at least one inflatable member has a partially circular cross-sectional shape. In other aspects, the at least one inflatable member has a generally rectangular cross-sectional shape.

In some implementations, the at least one inflatable member is configured to be inflated with a fluid, a gas, or a vapor.

In one aspect, the at least one inflatable member comprises a scraping element configured to remove thrombus or other biological materials from the vessel wall.

In some aspects, the at least one inflatable member is further configured to increase a stiffness of the funnel.

In one aspect, the device further includes at least one scraping element, the at least one inflatable member being configured to deploy the at least one scraping element from a collapsed configuration to a deployed configuration.

In some aspects, the at least one scraping element is configured to pivot around an attachment point to the funnel.

A method for removing a thrombus from a blood vessel of a patient is provided, the method comprising: introducing a distal portion of an elongate catheter in proximity to a thrombus location in a blood vessel; expanding a funnel distal to the distal portion in the blood vessel; drawing at least a section of the thrombus into the distal portion; expanding at least one inflatable member of the funnel to improve engagement between the thrombus and the funnel; and aspirating the thrombus into the elongate catheter.

In some aspects, the method includes directing fluid toward the thrombus with at least two different fluid streams. In another aspect, the method includes expanding at least one inflatable member of the funnel to increase a stiffness of the funnel.

In some aspects, the method includes expanding at least one inflatable member of the funnel to actuate a scraping element from a collapsed configuration to a deployed configuration.

In another aspect, the method includes removing thrombus or biological material from a vessel wall with the scraping element.

A method for removing a thrombus from a blood vessel of a patient is provided, the method comprising: introducing a distal portion of an elongate catheter in proximity to a thrombus location in a blood vessel; expanding a funnel distal to the distal portion in the blood vessel; drawing at least a section of the thrombus into the distal portion; and expanding at least one inflatable member of the funnel to improve engagement between a vessel wall and the funnel.

In one aspect, the method includes directing fluid toward the thrombus with at least two different fluid streams.

In other aspects the method includes expanding at least one inflatable member of the funnel to increase a stiffness of the funnel.

In one aspect, the method includes expanding at least one inflatable member of the funnel to actuate a scraping element from a collapsed configuration to a deployed configuration.

In another aspect, the method includes removing thrombus or biological material from a vessel wall with the scraping element.

A catheter device is provided, comprising: an elongate catheter shaft; a lumen disposed in the elongate catheter shaft; a funnel positioned at a distal portion of the elongate catheter shaft; and at least one inflatable member positioned on or in the funnel, the at least one inflatable member being configured to improve engagement between the funnel and a body lumen.

In some aspects, the at least one inflatable member is disposed on an interior surface of the funnel. In one aspect, the at least one inflatable member is disposed on an exterior surface of the funnel. In another aspect, the at least one inflatable member extends along a periphery of the funnel.

In some aspects, the at least one inflatable member comprises discrete inflatable members disposed at selected positions along the funnel.

In one aspect, the at least one inflatable member has a partially circular cross-sectional shape. In other aspects, the at least one inflatable member has a generally rectangular cross-sectional shape.

In some implementations, the at least one inflatable member is configured to be inflated with a fluid, a gas, or a vapor.

In one aspect, the at least one inflatable member comprises a scraping element configured to remove thrombus or other biological materials from the vessel wall.

In some aspects, the at least one inflatable member is further configured to increase a stiffness of the funnel.

In one aspect, the device further includes at least one scraping element, the at least one inflatable member being configured to deploy the at least one scraping element from a collapsed configuration to a deployed configuration.

In some aspects, the at least one scraping element is configured to pivot around an attachment point to the funnel.

### DETAILED DESCRIPTION

The present technology is generally directed to thrombus removal systems and associated methods. A system configured in accordance with an embodiment of the present technology can include, for example, an elongated catheter having a distal portion configured to be positioned within a blood vessel of the patient, a proximal portion configured to be external to the patient, a fluid delivery mechanism configured to fragment the thrombus with pressurized fluid, an aspiration mechanism configured to aspirate the fragments of the thrombus, and one or more lumens extending at least partially from the proximal portion to the distal portion.

The terminology used in the description presented below is intended to be interpreted in its broadest reasonable manner, even though it is being used in conjunction with a detailed description of certain specific embodiments of the present technology. Certain terms may even be emphasized below; however, any terminology intended to be interpreted in any restricted manner will be overtly and specifically defined as such in this Detailed Description section. Additionally, the present technology can include other embodiments that are within the scope of the examples but are not described in detail with respect to the figures.

Reference throughout this specification to “one embodiment” or “an embodiment” means that a particular feature, structure, or characteristic described in connection with the embodiment is included in at least one embodiment of the present technology. Thus, the appearances of the phrases “in one embodiment” or “in an embodiment” in various places throughout this specification are not necessarily all referring to the same embodiment. Furthermore, the particular features or characteristics may be combined in any suitable manner in one or more embodiments.

Reference throughout this specification to relative terms such as, for example, “generally,” “approximately,” and “about” are used herein to mean the stated value plus or minus 10%.

Although some embodiments herein are described in terms of thrombus removal, it will be appreciated that the present technology can be used and/or modified to remove other types of emboli that may occlude a blood vessel, such as fat, tissue, or a foreign substance. Additionally, although some embodiments herein are described in the context of thrombus removal from a pulmonary artery (e.g., pulmonary embolectomy), the technology may be applied to removal of thrombi and/or emboli from other portions of the vasculature (e.g., in neurovascular, coronary, or peripheral applications). Moreover, although some embodiments are discussed in terms of maceration of a thrombus with a fluid, the present technology can be adapted for use with other techniques for breaking up a thrombus into smaller fragments or particles (e.g., ultrasonic, mechanical, enzymatic, etc.).

The headings provided herein are for convenience only and do not interpret the scope or meaning of the claimed present technology.

### Systems for Thrombus Removal

As provided above, the present technology is generally directed to thrombus removal systems. Such systems include an elongated catheter having a distal portion positionable within a blood vessel of the patient (e.g., an artery or vein), a proximal portion positionable outside the patient's body, a fluid delivery mechanism configured to fragment the thrombus with pressurized fluid, an aspiration mechanism configured to aspirate the fragments of the thrombus, and one or more lumens extending at least partially from the proximal portion to the distal portion. In some embodiments, the systems herein are configured to engage a thrombus in a patient's blood vessel, break the thrombus into small fragments, and aspirate the fragments out of the patient's body. The pressurized fluid streams (e.g., jets) function to cut or macerate thrombus, before, during, and/or after at least a portion of the thrombus has entered the aspiration lumen or a funnel of the system. Fragmentation helps to prevent clogging of the aspiration lumen and allows the thrombus removal system to macerate large, firm clot that otherwise could not be aspirated. As used herein, “thrombus” and “embolism” are used somewhat interchangeably in various respects. It should be appreciated that while the description may refer to removal of “thrombus,” this should be understood to encompass removal of thrombus fragments and other emboli as provided herein.

According to embodiments of the present technology, a fluid delivery mechanism can provide a plurality of fluid streams (e.g., jets) to fluid apertures of the thrombus removal system for macerating, cutting, fragmenting, pulverizing and/or urging thrombus to be removed from a proximal portion of the thrombus removal system. The thrombus removal system can include an aspiration lumen extending at least partially from the proximal portion to the distal portion of the thrombus removal system that is adapted for fluid communication with an aspiration pump (e.g., vacuum source). In operation, the aspiration pump may generate a volume of lower pressure within the aspiration lumen near the proximal portion of the thrombus removal system, urging aspiration of thrombus from the distal portion.

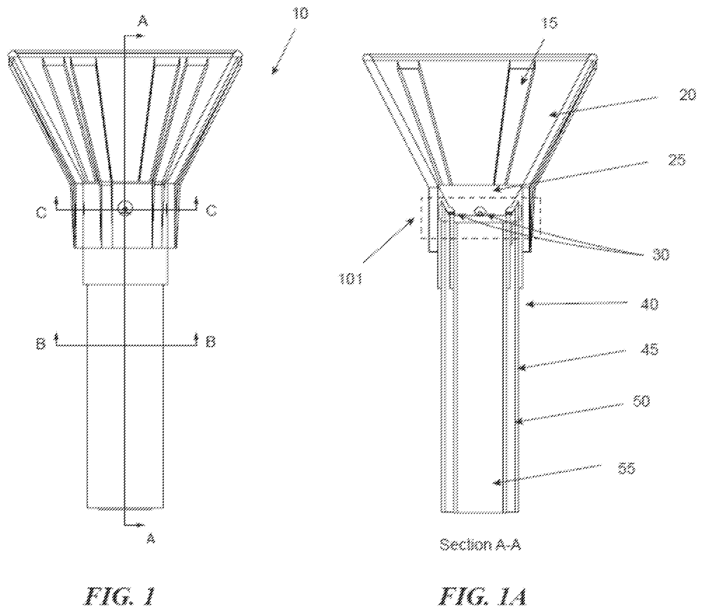

FIG. 1 illustrates a distal portion 10 of a thrombus removal system according to an embodiment of the present technology. FIG. 1A Section A-A illustrates an elevation sectional view of the distal portion. The example section A-A in FIG. 1A depicts a funnel 20 that is positioned at the distal end of the distal portion 10, the funnel adapted to engage with thrombus and/or a tissue (e.g., vessel) wall to aid in thrombus fragmentation and/or removal. The funnel can include an internal frame structure 15 that can provide structural support and/or expandability of the funnel, for example. The example section A-A in FIG. 1A depicts a double walled thrombus removal device construction having an outer wall/tube 40 and an inner wall/tube 50. An aspiration lumen 55 is formed by the inner wall 50 and is centrally located. A generally annular volume forms at least one fluid lumen 45 between the outer wall 40 and the inner wall 50. The fluid lumen 45 is adapted for fluid communication with a fluid delivery mechanism. In some embodiments, the fluid lumen 45 can further be adapted for fluid communication with one or more inflatable members of the thrombus removal device. Alternatively, the lumen 45 can be divided into separate fluid and inflation lumens. One or more apertures (e.g., nozzles, orifices, or ports) 30 are positioned in the thrombus removal system to be in fluid communication with the fluid lumen 45 and an irrigation manifold 25. In operation, the ports 30 are adapted to direct (e.g., pressurized) fluid toward thrombus that is engaged with the distal portion 10 of the thrombus removal system. In some embodiments, the manifold can be positioned proximal to the funnel. In other embodiments, the manifold can be integrated into the funnel.

The thrombus removal system can be sized and configured to access and remove thrombi in various locations or vessels within a patient's body. It should be understood that while the dimensions of the system may vary depending on the target location, generally the same features and components described herein will be implemented in the thrombus removal system regardless of the application. For example, a thrombus removal system configured to remove pulmonary embolism (PE) from a patient may have an outer wall/tube with a size of approximately 11-13 Fr, or preferably 12 Fr, and an inner wall/tube with a size of 7-9 Fr, or preferably 8 Fr. A deep vein thrombosis (DVT) device, on the other hand, may have an outer wall/tube with a size of approximately 9-11 Fr, or preferably 10 Fr, and an inner wall/tube with a size of 6-9 Fr, or preferably 7.5 Fr. Applications are further provided for ischemic stroke and peripheral embolism applications.

In some embodiments, the ports are formed to direct the fluid flow along a selected path. In some embodiments, at least two ports are arranged to produce (e.g., respective) fluid streams that intersect at an intersection region of the thrombus removal system. An intersection region can be a region of increased fluid momentum and/or energy transfer, which increase is with respect to individual fluid streams that are not directed to combine at the intersection. The increased fluid momentum and/or energy transfer at an intersection may advantageously fragment thrombus more efficiently and/or quickly. In some embodiments, an intersection region can be formed from at least 2, at least 3, at least 4, at least 5, at least 6, at least 7, at least 8, at least 9, or at least 10 fluid streams. An intersection region can be generally near a central axis of the thrombus removal system, or away from the central axis. In some embodiments, at least two intersection regions are formed. In some embodiments, one or more are arranged to direct a fluid stream along an oblique angle with respect to the central axis of the thrombus removal system. An operating pressure of the fluid delivery mechanism may be selected to approach a targeted fluid velocity for a fluid stream that is delivered from a port. In some embodiments, at least two ports are adapted to deliver respective fluid streams at different fluid velocities, for a given pressure of the fluid deliver mechanism. In some embodiments, at least two ports are adapted to deliver respective fluid streams at the substantially the same fluid velocities, for a given pressure of the fluid delivery mechanism. In some embodiments, angular momentum is imparted to a thrombus by application of a) at least one fluid stream that is directed at an oblique angle from a port, and/or b) at least two fluid streams that have different fluid velocities. Advantageously, angular momentum produced in a thrombus may impart a (e.g., centrifugal) force that assists in fragmentation and removal of the thrombus. Advantageously, an increased cross-sectional area of the fluid lumen reduces a required operating pressure of the fluid delivery mechanism to achieve a targeted fluid velocity of the fluid streams.

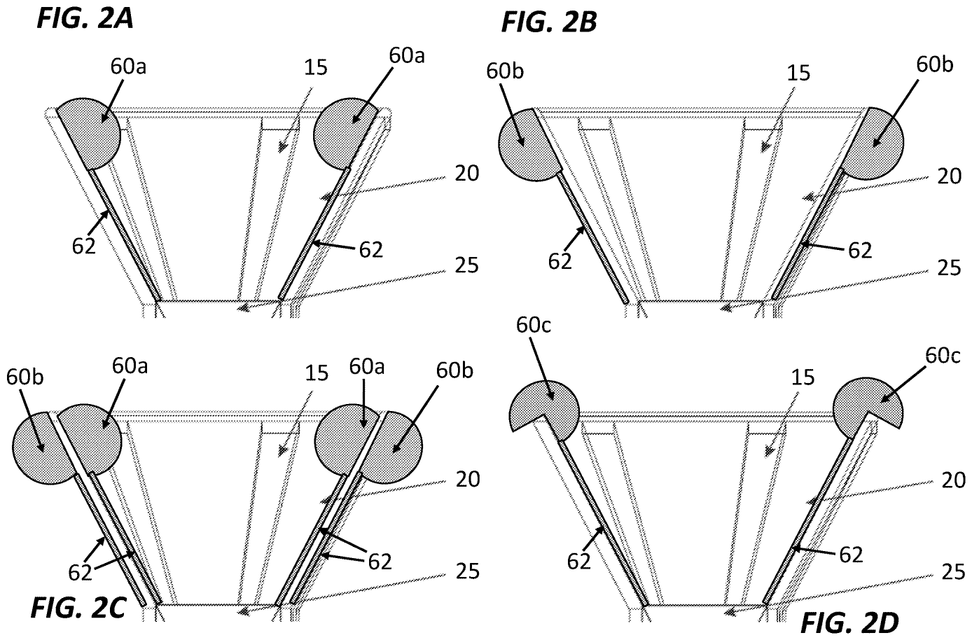

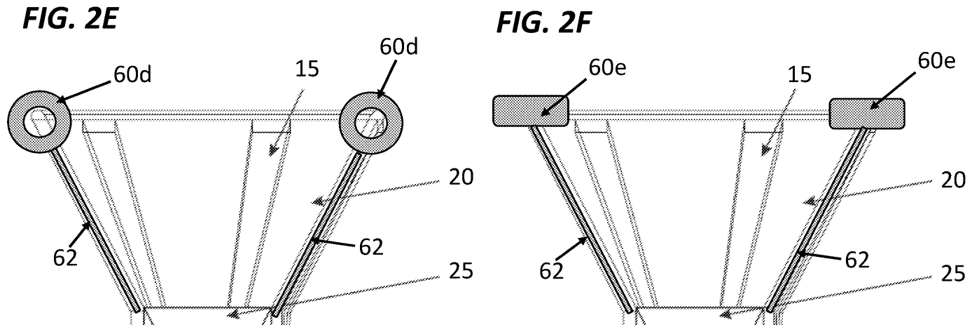

FIGS. 2A-2F illustrate embodiments of an inflatable member or member(s) (60a, 60b, 60c, 60d, 60e) disposed on or within the funnel 20. The inflatable member(s) can be configured to improve engagement between the funnel and one or more thrombi in or near the funnel. Additionally, the inflatable member(s) can be configured to improve engagement between the funnel and a vessel wall adjacent to the funnel. In some embodiments, the inflatable member(s) increase engagement with both the thrombus/thrombi and the vessel wall. The inflatable member(s) can be fluidly coupled to an inflation lumen and/inflation system with inflation lumens 62 that can be configured and adapted to inflate/deflate the inflation member(s) when needed. In some embodiments, the inflation member(s) can be inflated with a gas, such as air. In other embodiments, the inflation member(s) can be inflated with a fluid, such as saline, water, or a vapor such as steam.

Referring to FIG. 2A, the thrombus removal device can include one or more inflatable members 60a disposed around a periphery of the funnel 20. In this embodiment, the inflatable member(s) 60a is generally circular in cross-section and is disposed on an inner edge or wall of the funnel 20. The inflatable members 60a can be fluidly coupled to the inflation system (not shown) with inflation lumens 62. In this embodiment, with the engagement member(s) 60a disposed in the interior of the funnel, the inflatable member(s) 60a can be configured to improve engagement with at least one thrombus positioned in or near the funnel. For example, if the thrombus is partially engaged with the funnel (i.e., engaged with one or more interior portions of the funnel), then the engagement member(s) 60a can be inflated with a gas, fluid, or vapor to expand and contact or provide better engagement with the thrombus or thrombi. In some examples, when the clot engages with the inflatable member(s) it can cause the funnel to “bow out” or collapse proximally (pulled towards morcellation plane) which brings the clot or thrombus towards the aspiration lumen In some embodiments, the engagement member(s) 60a can be disposed around an entirety of the periphery of the funnel (e.g., an annular balloon or inflatable member). In other embodiments, the inflatable member(s) 60a can be multiple discrete inflatable members disposed at selection positions around the funnel.

FIG. 2B illustrates a thrombus removal device having one or more inflatable members 60b disposed around a periphery of the funnel 20. However, in this embodiment, the inflatable member(s) 60b are disposed on the exterior of the funnel. In this embodiment, the inflatable member(s) 60b can be configured to improve engagement between the funnel and a vessel wall. For example, it may be desirable to fully engage a periphery of a vessel wall with the funnel. In some situations, a fully expanded funnel may not achieve this goal. The inflatable members 60b can be fluidly coupled to the inflation system (not shown) with inflation lumens 62. Therefore, the one or more inflatable member(s) 60b can be inflated to increase a diameter of the funnel to better or more fully engage the vessel wall. In some embodiments, the engagement member(s) 60b can be disposed around an entirety of the periphery of the funnel (e.g., an annular balloon or inflatable member). In other embodiments, the inflatable member(s) 60b can be multiple discrete inflatable members disposed at selection positions around the funnel.

In some examples, the inflatable members 60b positioned on the exterior of the funnel can be used as vessel wall scrapers. For example, the inflatable members 60b can be expanded and configured to scrape or engage with materials such as clots or plaque located on an interior wall of a vessel or interior lumen of the body. In some embodiments, the inflatable members can comprise a material suitable for scraping materials from an interior vessel wall or lumen. The inflatable members can additionally be shaped to improve scraping or engagement with vessel substances (i.e., concave or pointed surfaces configured to engage with and remove clots or plaque from vessel walls).

In some embodiments, the thrombus removal device can include both inflatable member(s) configured to improve engagement with a thrombus on the interior of the funnel and inflatable member(s) configured to improve engagement with a vessel on the exterior of the funnel. For example, referring to FIG. 2C, the thrombus removal device can include both interior inflatable member(s) 60a and exterior inflatable member(s) 60b. In some embodiments, the inflatable member(s) 60a and 60b can be inflated independently. The inflatable members 60a and 60b can be fluidly coupled to the inflation system (not shown) with separate inflation lumens 62. For example, if better engagement with a thrombus is desired, only inflatable member(s) 60a can be inflated. If only better engagement with a vessel wall is desired, only inflatable member(s) 60b can be inflated. In one embodiment, inflatable members 60b can be uninflated but still extend as a skirt. When member 60b is un-inflated may allow the vacuum to more effectively pull the clot to the morcellation plane. Furthermore, both inflatable member(s) 60a and 60b can be inflated to improve engagement with both. In some embodiments, the engagement member(s) 60b can be disposed around an entirety of the periphery of the funnel (e.g., an annular balloon or inflatable member). In other embodiments, the inflatable member(s) 60b can be multiple discrete inflatable members disposed at selection positions around the funnel.

While the embodiment of FIG. 2C includes separate interior and exterior inflatable members, the embodiment of FIG. 2D includes one or more inflatable member(s) 60c that can perform both functions of improving engagement with a thrombus in the interior of the funnel and improving engagement with a vessel wall exterior to the funnel. The inflatable member(s) can include any number of cross-sectional shapes, including the partially circular shape of inflatable member(s) 60c in FIG. 2D, the “donut” shaped cross-section of inflatable member 60d in FIG. 2E, or any number of flat or rectangular shaped cross-sections in inflatable member 60e of FIG. 2F. The inflatable members 60c, 60d, and 60e can be fluidly coupled to the inflation system (not shown) with inflation lumens 62.

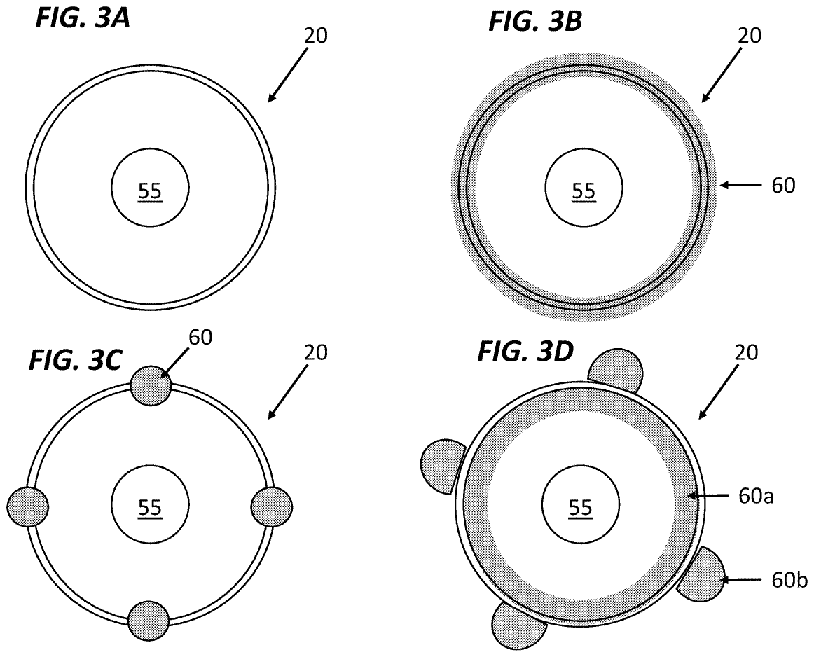

FIGS. 3A-3D illustrate top down views of a thrombus removal device, including a funnel 20 and one or more inflatable member(s) 60. FIG. 3A shows a funnel 20 with the inflatable member(s) 60 deflated. In this embodiment, only the funnel itself is configured to contact or engage a thrombus and/or a vessel wall. FIG. 3B shows a funnel 20 with an annular shaped inflatable member 60 that extends around the perimeter of the funnel. In this embodiment, it should be understood that the inflatable member can include any cross-sectional shape, including but not limited to any of the cross-sectional shapes shown in FIGS. 2A-2F. It should also be understood that the inflatable members of 60 can be fluidly coupled to an inflation system with one or more inflation lumens (not shown, but previously illustrated and described above).

In the embodiment of FIG. 3C, the thrombus removal device includes a funnel and a plurality of discrete inflatable members 60. In this example, the inflatable members do not extend around the entire periphery of the funnel, but instead are placed at selected locations along the funnel. As with above, the inflatable members can include any cross-sectional shape, including but not limited to the examples provided herein.

In the embodiment of FIG. 3D, the thrombus removal device includes a funnel and both an annular inflatable member 60a that extends around a periphery of the funnel, but also includes a plurality of discrete inflatable members 60b. In this example, the annular inflatable member 60a is on the interior of the funnel, and the plurality of discrete inflatable members are disposed on an exterior of the funnel. However, it should be understood that any combination of annular/discrete inflatable members can be implemented. Additionally, it should be understood that the annular inflatable member need not extend fully around the periphery of the funnel. In some examples, the annular inflatable member can extend up to 25%, up to 50%, up to 75%, or up to substantially 99% of the periphery of the funnel. In some examples, two or more annular inflatable members can be implemented. It should also be understood that the inflatable member(s) 60, 60a, or 60b of FIGS. 3A-3D can be configured to engage with a vessel wall to scrape or remove materials from the vessel wall, such as clot or plaque.

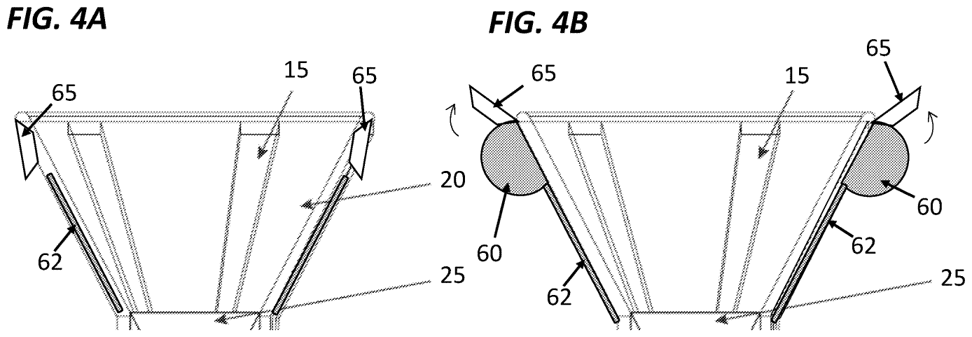

FIGS. 4A-4B illustrate another embodiment where inflatable members 60 of a funnel 20 can be configured to actuate or support separate scraping elements 65. In FIG. 4A, the inflatable members 60 are deflated, allowing scraping elements 65 to be in a collapsed or un-actuated position. In some embodiments, the collapsed position allows the scraping elements to rest against the funnel or optionally rest within the funnel (such as within cutouts that conform to the shape of the scraping elements). In FIG. 4B, the inflatable members 60 have been inflated via inflation lumens 62, causing actuation of the scraping elements 65 into a deployed position. The inflatable members are configured to act as a support for the scraping elements. In some implementations, the inflation level of the inflatable members can be metered or adjusted to fine tune the amount of force applied by the scraping elements to a vessel wall. In some embodiments, inflation of the inflatable members can cause the scraping elements to pivot or rotate about an attachment point to the funnel, as shown and indicated by the arrows in FIG. 4B. The amount of inflation can determine the degree of deployment of the scraping elements. For example, more inflation can cause the scraping elements to rotate or pivot to a greater angle. The angle of deployment can therefore be adjusted depending on the anatomy and the amount of scraping or wall engagement desired. In other embodiments, the scraping elements can instead be fixed or attached to the inflatable members themselves, and inflation of the inflatable members causes the scraping elements to be moved into place.

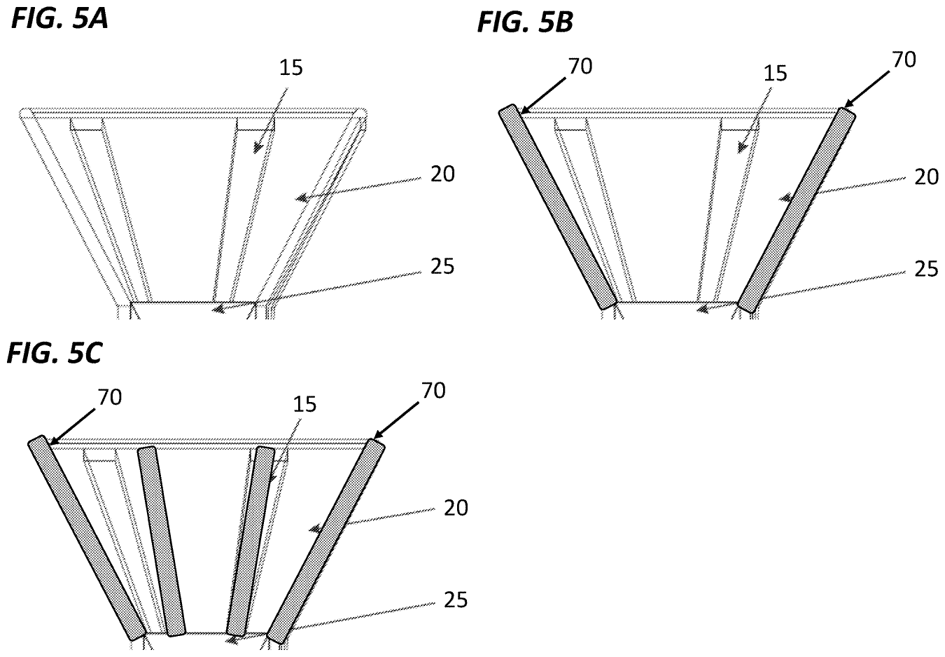

In additional embodiments, referring to FIGS. 5A-5C, inflatable members 70 can instead be used to rigidize the funnel. FIG. 5A shows a funnel 20 with one or more frame structure elements 15, as previously described. In this embodiment, the inflatable members can be deflated, and the stiffness and/or compliance of the funnel is determined by the frame structure elements 15 and the membrane or compliant material used for the funnel itself. However, it may be desirable to increase the stiffness of the funnel, or apply stiffness to specific regions of the funnel. Therefore, referring to FIGS. 5B-5C, embodiments are provided that include inflatable members 70 which can be selectively inflated/deflated with an inflation system to adjust the stiffness of the funnel. In some examples, the inflatable members 70 can extend axially along a length of the funnel, as shown in FIG. 5B. In one embodiment, the entirety of the funnel can include a conically shaped inflatable member (FIG. 5B) that can be inflated to increase stiffness of the funnel. In another embodiment, referring to FIG. 5C, a plurality of expandable members 70 can be disposed throughout the funnel. The inflatable members can extend in an axial direction of the funnel, a radial direction of the funnel, or both. In some embodiments, the inflatable members are positioned alongside or near the frame structure elements 15. In other embodiments, the inflatable members are disposed away from the frame structure elements to provide optional stiffness in areas of the funnel that would otherwise be compliant. In some embodiments, the inflatable members 70 used for internal tension/rigidity of the funnel can have an inflation pressure higher than the inflatable members that are shown, for example, in FIGS. 2A-2F that are used for clot/wall engagement.

The inflatable members can be inflated with gas or fluid, such as air or saline. Any other suitable inflation mediums are contemplated and within the scope of this disclosure. In some embodiments, the inflatable members can be automatically inflated by one or more processors or controllers of the thrombectomy system. For example, the inflatable members may be automatically inflated when the funnel is engaged with a thrombus, to make the funnel stiffer while attempting to break up and aspirate the thrombus.

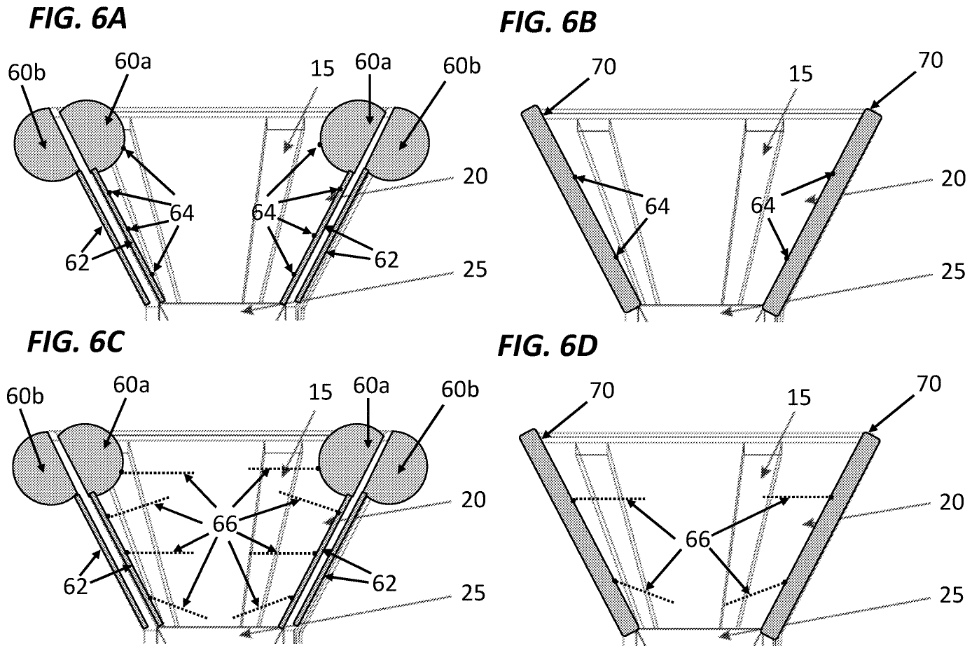

In some embodiments, any of the inflation lumens 62 and/or inflatable members 60, 60a, 60b, 60c, 60d, 60e, or 70 of the devices and systems described herein can include apertures or ports 64 configured to deliver a fluid stream or into the funnel of the thrombus removal device. The fluid streams/jets can be configured to break up, cut, or macerate thrombi or clots disposed within or engaged with the funnel. Any of the embodiments described herein can include apertures/ports/jets. Referring to FIGS. 6A-6B, ports 64 can be disposed in the inflation lumens 62 and/or in the inflatable members 60a/60b/70. One or more ports can be disposed in the inflation lumens or members, as shown. FIGS. 6C to 6D show fluid streams 66 being delivered from the ports into the funnel. In some embodiments, fluid streams on one side of the funnel can be configured to cross or intersect with one or more fluid streams from the other side of the funnel. The fluid streams can be directed orthogonal to a longitudinal axis of the device, be directed proximally, be directed distally, or any combination thereof. The fluid streams can comprise the same fluid used to inflate the inflatable members (e.g., saline). It should be understood that any inflation medium can be used to produce the fluid streams.

When incorporating ports and fluid streams/jets into the inflatable members and/or inflation lumens, it should be understood that there can be an inflation pressure that is less than the jet/fluid stream pressure. Therefore, there will be a low flow rate of fluid out of the ports when the inflatable members are inflated but jetting/fluid streams are not desired. When fluid streams/jetting is turned on, the pressure inside the inflation lumens and inflatable members is increased. Since the inflatable members are already stiff when inflated to in the inflation pressure, the internal volume of the inflatable members does not change significantly when the pressure is increased to the jet/fluid stream pressure. This supports a rapid increase in velocity of the fluid which makes the fluid streams/jets more penetrating (such as into a clot or thrombus within the funnel).

The above detailed description of embodiments of the technology are not intended to be exhaustive or to limit the technology to the precise forms disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

Unless the context clearly requires otherwise, throughout the description and the examples, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. As used herein, the phrase “and/or” as in “A and/or B” refers to A alone, B alone, and A and B. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

1. A thrombus removal device, comprising:
an elongate catheter shaft;
an aspiration lumen disposed in the elongate catheter shaft;
a funnel positioned at a distal portion of the elongate catheter shaft and configured to engage a thrombus and/or a vessel wall;
at least two fluid ports formed in the distal portion and adapted to direct fluid streams from the at least two fluid ports;
at least one inflatable member positioned on or in the funnel, the at least one inflatable member being configured to improve engagement with the thrombus and/or vessel wall.

2. The device of claim 1, wherein the at least one inflatable member is disposed on an interior surface of the funnel.

3. The device of claim 1, wherein the at least one inflatable member is disposed on an exterior surface of the funnel.

4. The device of claim 1, wherein the at least one inflatable member extends along a periphery of the funnel.

5. The device of claim 1, wherein the at least one inflatable member comprises discrete inflatable members disposed at selected positions along the funnel.

6. The device of claim 1, wherein the at least one inflatable member has a partially circular cross-sectional shape.

7. The device of claim 1, wherein the at least one inflatable member has a generally rectangular cross-sectional shape.

8. The device of claim 1, wherein the at least one inflatable member is configured to be inflated with a fluid, a gas, or a vapor.

9. The device of claim 1, wherein the at least one inflatable member comprises a scraping element configured to remove thrombus or other biological materials from the vessel wall.

10. The device of claim 1, wherein the at least one inflatable member is further configured to increase a stiffness of the funnel.

11. The device of claim 1, further comprising at least one scraping element, the at least one inflatable member being configured to deploy the at least one scraping element from a collapsed configuration to a deployed configuration.

12. The device of claim 1, wherein the at least one scraping element is configured to pivot around an attachment point to the funnel.

13. A method for removing a thrombus from a blood vessel of a patient, the method comprising:
introducing a distal portion of an elongate catheter in proximity to a thrombus location in a blood vessel;
expanding a funnel distal to the distal portion in the blood vessel;
drawing at least a section of the thrombus into the distal portion;
expanding at least one inflatable member of the funnel to improve engagement between the thrombus and the funnel; and
aspirating the thrombus into the elongate catheter.

14. The method of claim 13, further comprising directing fluid toward the thrombus with at least two different fluid streams.

15. The method of claim 13, further comprising expanding at least one inflatable member of the funnel to increase a stiffness of the funnel.

16. The method of claim 13, further comprising expanding at least one inflatable member of the funnel to actuate a scraping element from a collapsed configuration to a deployed configuration.

17. The method of claim 16, further comprising removing thrombus or biological material from a vessel wall with the scraping element.

18. A method for removing a thrombus from a blood vessel of a patient, the method comprising:
introducing a distal portion of an elongate catheter in proximity to a thrombus location in a blood vessel;
expanding a funnel distal to the distal portion in the blood vessel;
drawing at least a section of the thrombus into the distal portion;
expanding at least one inflatable member of the funnel to improve engagement between a vessel wall and the funnel.

19. The method of claim 18, further comprising directing fluid toward the thrombus with at least two different fluid streams.

20. The method of claim 18, further comprising expanding at least one inflatable member of the funnel to increase a stiffness of the funnel.

21. The method of claim 18, further comprising expanding at least one inflatable member of the funnel to actuate a scraping element from a collapsed configuration to a deployed configuration.

22. The method of claim 21, further comprising removing thrombus or biological material from a vessel wall with the scraping element.

23.-33. (canceled)
